(19) United States
(12) Patent Application Publication
(10) Pub. No.: US 2006/0052819 A1
(43) Pub. Date: Mar. 9, 2006

(54) Surgery fixation forceps

(76) Inventor: Yen-Yue Lin, Taipei (TW)

Correspondence Address: LEONG C LEI, PMB # 1008, 1867 YGNACIO VALLEY ROAD, WALNUT CREEK, CA, 94598, US

(21) Appl. No.: 11/042,131

(22) Filed: Jan. 26, 2005

(30) Foreign Application Priority Data
Sep. 7, 2004 (TW) 093214216

(51) Int. Cl. A61B 17/50

(52) U.S. Cl. 606/211

(57) ABSTRACT

A surgery forceps also functioning as a surgery scissors used in suture operation to shorten time required for suture, anesthesia, and anesthetic dosage, cut down the number of attending nurse, and reduced consumption of operation appliances, is essentially comprised of a pillar blade holder provided at the top of the forceps; a blade contained in the holder to cut of the suture upon completing the suture operation simply by turning the forceps up side down without putting down the forceps to fetch for the scissors or waiting for the nurse to cut off the suture.

### BACKGROUND OF THE INVENTION

(a) Technical Field of the Invention

The present invention is related to a pair of surgery fixation forceps, and more particularly to one that also operates as a pair of ligature cutting scissors in suture operation to cut suture lines.

(b) Description of the Prior Art

Referring to FIG. 1 of the accompanying drawings, in the skin suture for example, the doctor has one hand holding a pair of fixation forceps 1 to hold the peripheral skin to a wound 11, a lacerated wound in this case, and another hand holding a pair of ligature forceps 2. A needle 21 is sandwiched between the tips of the ligature forceps 2 and connected with a suture 22. Stitch by stitch in sequence, the doctor completes the suture operation for the patient. The problem is that upon completing each stitch, the suture 22 must be cut off using a pair of ligature cutting scissors 3 before proceeding to the next stitch. Therefore, upon completing a stitch, the doctor has to spare one hand to fetch for the ligature cutting scissors 3 to cut of the suture, or a nurse must be available to do the cutting for the doctor making the entire suture operation not meeting economic benefits in either way.

### SUMMARY OF THE INVENTION

The primary purpose of the present invention is to provide a pair of surgery fixation forceps also functioning as a pair of ligature cutting scissors. The doctor simply turn the fixation forceps up side down to do the cutting without fetching for another cutting scissors or having the nurse to do the cutting. To achieve the purpose, a pillar holder inserted with a blade is disposed at the top of the fixation forceps. The present invention provides the following benefits:
1. Shorten the time required for suture and anesthesia, and reduced anesthetic dosage;
2. Fewer nurses are required to attend the operation; and
3. Reduced use or replacement of surgery appliances.

Another purpose of the present invention is to provide a pair of fixation forceps also functioning as a pair of ligature cutting scissors that is safe to use without causing injury to the patient by accident. To achieve the purpose, the blade inserted to the pillar holder protruding from the top of the fixation forceps is provided with two arc top edges to define an open slot and a blade is provided at the bottom edge of the slot. Accordingly, upon completing the suture operation, the free end of the suture is slightly pulled up and then pushing forward the fixation forceps to use the blade in the slot to hold against the suture and easily cut off the suture. The blade provides the following advantages:
1. Facilitate cutting off the suture;
2. Not hurting the patient or the doctor, and the skin; and
3. Applicable to subcutaneous suture.

Another purpose yet of the present invention is to provide a pair of fixation forceps also functioning as a pair of ligature cutting scissors that may be used in replacement of a general surgery knife. To achieve the purpose, the blade inserted in the blade holder protruding from the top of the fixation forceps relates to a long blade with a variant blade.

Another purpose yet of the present invention is to provide a pair of fixation forceps also functioning as a pair of ligature cutting scissors that is further provided with a retractable blade holder. To achieve the purpose, an insertion opening to the blade holder for the holder to be inserted to the top of the fixation forceps and held in position of an adjustable screw.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a schematic view showing multiple appliances are used in a suture operation.

FIG. 2 is a perspective view of a preferred embodiment of the present invention.

FIG. 3 is a schematic view showing an assembly of the preferred embodiment of the present invention.

FIG. 4 is a schematic view showing that another type of blade is inserted to a pillar blade holder of the present invention of the present invention.

FIG. 5 is a perspective view of another preferred embodiment of the present invention.

FIG. 6 is a schematic view of another preferred embodiment of the present invention as assembled.

FIG. 7 is a schematic view showing the operation of the present invention.

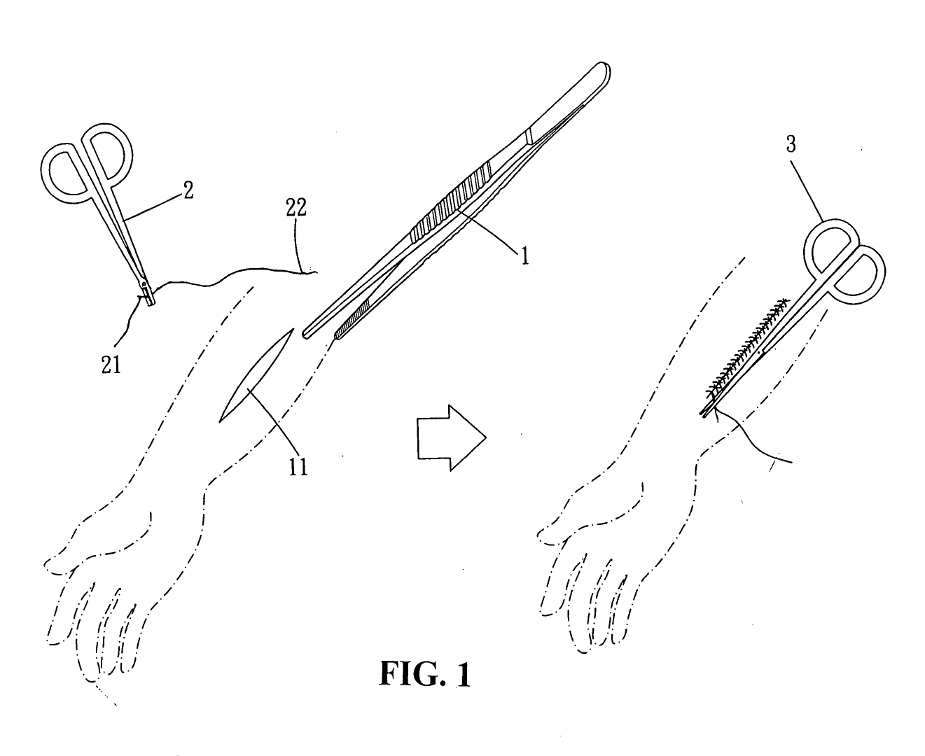

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

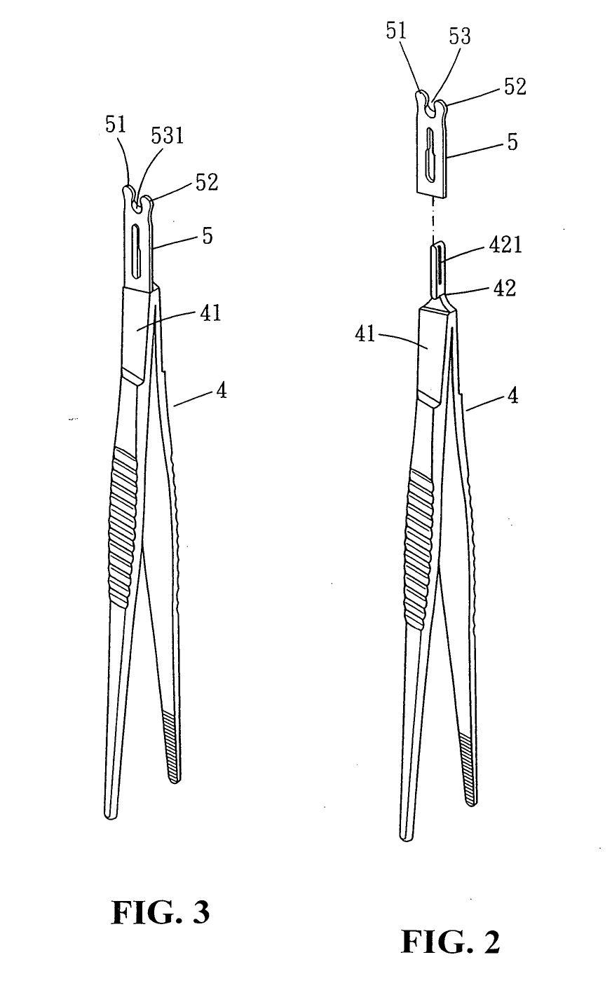

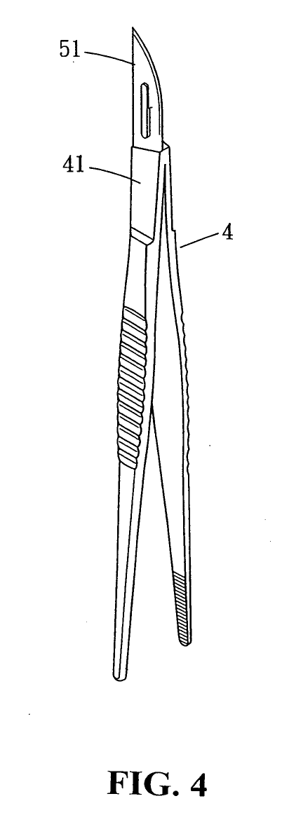

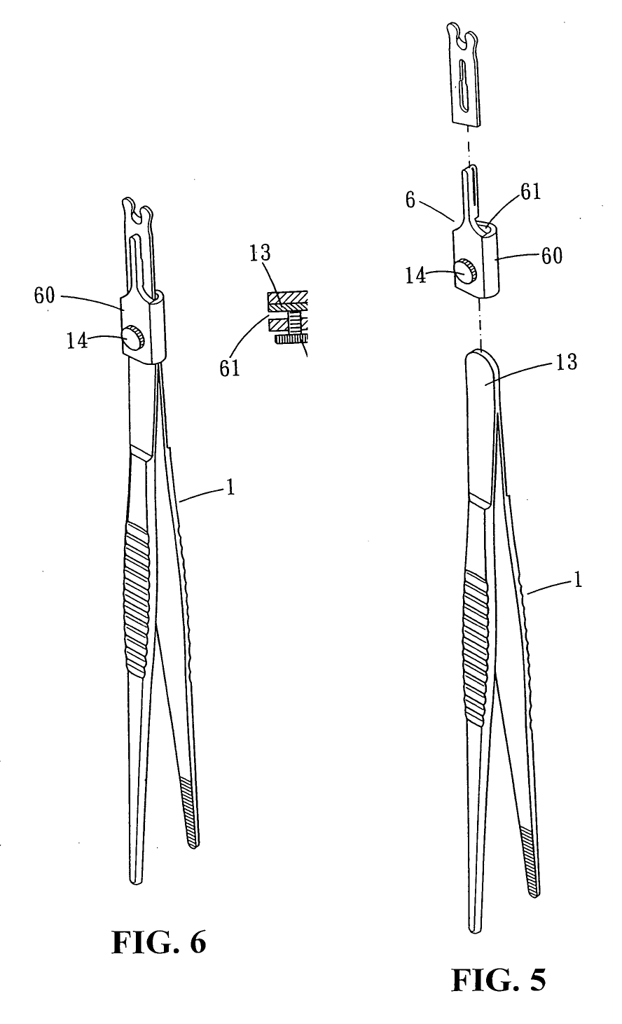

Referring to FIGS. 2 and 3 for a preferred embodiment of the present invention, a pair of fixation forceps 4 has at its top 41 provided with a pillar blade holder 42. A channel 421 is axially provided to the blade holder 42 to secure the insertion of a blade 5 to cut off the suture. Wherein, the blade 5 is provided at its top two ears 51, 52 to define an open slot 53 with a blade 531 disposed at the bottom of the slot 53. Upon completing the suture operation, the pair of the fixation forceps 4 is pushed forward for the slot 53 to catch one free end of the suture and to cut it off with the blade 531. As illustrated in FIG. 5, the present invention is also used as a conventional surgery knife. Wherein, the blade relates to a long blade 50 with a variant blade to be inserted into the holder protruding from the top 41 of the fixation forceps 4.

Now referring to FIG. 5 for another preferred embodiment of the present invention, a pair of fixation forceps 1 generally used by the surgery doctors is provided an insertion opening 61 at the lower part 60 of a blade holder 6 for the insertion of the blade. Atop section 13 of the fixation forceps 1 is inserted into and confines the pillar blade holder 6 through the insertion opening 61. An adjustable screw 14 is provided on the other side of the holder 6 opposite to where the insertion opening 61 is disposed to secure the holder to the top section of the fixation forceps 1. Accordingly, the blade holder is retractable from the fixation forceps 1.

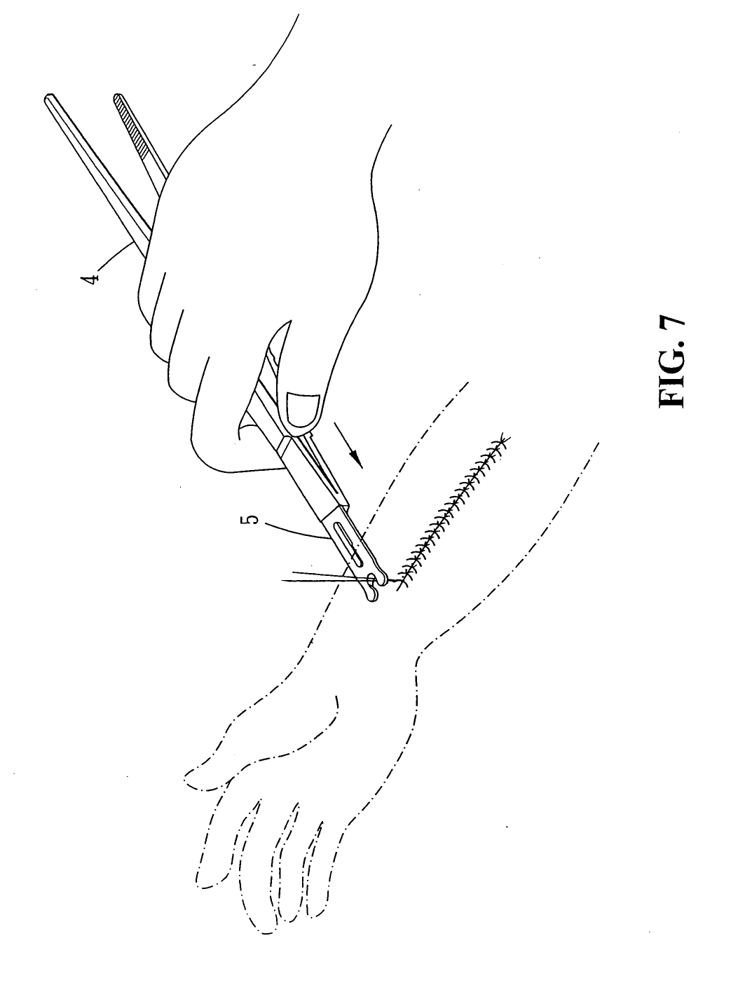

As illustrated in FIG. 7, the fixation forceps also functioning as a ligature cutting scissors is pushed forward to cut off the free end of a suture.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A pair of surgery fixation forceps also functioning as a pair of ligature cutting scissors is essentially comprised of a pillar blade holder protruding from the top of the forceps; and a blade is inserted and secured in the holder to cut off the free end of a suture simply by turning the forceps up side down.

2. The pair of surgery fixation forceps also functioning as a pair of ligature cutting scissors of claim 1, wherein two ears are extended from the top of the blade inserted and secured in the pillar holder protruding from the top of the forceps to define a slot; and a blade of the blade is disposed at the bottom of the slot.

3. The pair of surgery fixation forceps also functioning as a pair of ligature cutting scissors of claim 1 or 2, wherein the blade relates to a long blade with a variant blade to function as a conventional surgery knife.

4. The pair of surgery fixation forceps also functioning as a pair of ligature cutting scissors of claim 1, wherein the blade holder is retractable; an insertion opening is provided to the pillar blade holder; the holder is inserted to the top section of the forceps through the insertion opening; and an adjustable screw is provided on the side of the holder opposite to where the insertion opening is disposed for the holder.
